(19) United States
(12) Patent Application Publication
(10) Pub. No.: US 2013/0157858 A1
(43) Pub. Date: Jun. 20, 2013

(54) METHODS AND SYSTEMS USEFUL FOR CONTROLLING INVASIVE WATERMILFOIL

(71) Applicants: Mark A. Heilman, Carmel, IN (US); Ryan Thum, Muskegon, MI (US)

(72) Inventors: Mark A. Heilman, Carmel, IN (US); Ryan Thum, Muskegon, MI (US)

(21) Appl. No.: 13/407,064

(22) Filed: Feb. 28, 2012

Related U.S. Application Data
(60) Provisional application No. 61/447,347, filed on Feb. 28, 2011.

(51) Int. Cl. C12Q 1/68; G06N 5/02

(52) U.S. Cl. CPC C12Q 1/6895; G06N 5/02
USPC 504/155; 504/157; 435/6.11; 706/46

(57) ABSTRACT

Described are methods and systems for managing watermilfoil populations in bodies of water such as lakes. In certain forms, the methods and systems involve the use of genetic analysis to determine whether a watermifoil population targeted for herbicidal control has a genetic relationship with hybrid (e.g. Northern×Eurasian) watermilfoil plants known to exhibit herbicidal tolerance. In one aspect, it has for the first time been discovered that such hybrid watermilfoil plants include significant clustering groups that exhibit herbicide tolerance. This can be used in the design and implementation of herbicidal treatment regimens against a new target watermilfoil population.

### REFERENCE TO RELATED APPLICATION

This application claims the benefit of U.S. Provisional Patent Application Ser. No. 61/447,347 filed Feb. 28, 2011, which is hereby incorporated herein by reference in its entirety.

### BACKGROUND

The present invention relates generally to weed control, and in certain embodiments to methods and systems for controlling watermilfoil plants in lakes or other water bodies using chemical herbicides.

As further background, various methods exist for the control of aquatic weeds such as watermilfoil. The selection of an appropriate control method depends upon many factors such as environmental impact, cost effectiveness, efficacy, and the like. The various control methods available include physical controls such as mechanical harvesting, hand pulling or cutting, or the use of bottom barriers or water level draw-down. These methods can be both time consuming and labor intensive, and can create significant environmental disturbance, especially when considered on a large scale. Other mechanical controls such as rotovation have similar drawbacks.

Biological controls such as the use of organisms that feed on aquatic weeds can be desirable in some aquatic systems in that they reduce the use of equipment and have the potential for long term control. In temperate aquatic systems, the efficacy of such biological controls can also vary widely, and is dependent upon factors such as feeding preferences, metabolism, temperature, and stocking rate.

For these and other related reasons, the use of aquatic herbicides has become a common method for controlling invasive aquatic weeds. The use of herbicidal control nonetheless also presents risks and difficulties including the potential impact on the local environment, the potential for excessive decrease in the dissolved oxygen (DO) content of the waters due to rapid plant decay, and the potential for tolerance development, especially where an effective elimination of an invasive organism is not achieved during a treatment.

In light of this background, there is a need for improved methods for the control of aquatic weeds, including watermilfoil, with chemical herbicides. Such methods would desirably facilitate successful control of the target weed or weeds. The present invention addresses these needs.

### SUMMARY

In one embodiment, the invention provides a method for controlling watermilfoil in a lake. The method includes obtaining samples of target watermilfoil plants living in a lake, and obtaining genetic information from the samples. A relationship of the target watermilfoil plants to other watermilfoil plants, for example a phylogenetic or other biotype-grouping relationship, is inferred from the genetic information, wherein at least some of the other watermilfoil plants have confirmed tolerance to at least one chemical herbicidal agent. The method further includes treating the body of water with a selected chemical herbicidal agent in accordance with a treatment regimen based at least in part upon said inferring step.

In another embodiment, the invention provides a computer-based system useful for inferring a genetic relationship, for example a phylogenetic or other biotype-group relationship, of a target watermilfoil plant to other watermilfoil plants. The system includes a processing unit and one or more memory storage units coupled to the processing unit. The one or more memory storage units store (i) a routine for estimating a genetic relationship, for example for estimating a phylogenetic tree, (ii) first data representing genetic information from the target watermilfoil plant, and (iii) second data representing genetic information from multiple genetically (e.g. phylogenetically) differing watermilfoil plants and associating confirmed chemical herbicidal tolerance with at least some of said multiple differing watermilfoil plants. The routine, first data and second data are processable by the processing unit to estimate a genetic relationship, for example in the form of a phylogenetic tree, including the target watermilfoil plant and the multiple genetically differing watermilfoil plants.

In another embodiment, the invention provides a method for controlling target watermilfoil plants in a body of water. The method includes treating the body of water with a selected chemical herbicidal agent in accordance with a treatment regimen, wherein the treatment regimen has been based at least in part upon inferring from genetic information from the target watermilfoil plant a genetic relationship of the target watermilfoil plants to other watermilfoil plants. At least some of the other watermilfoil plants have confirmed tolerance to at least one chemical herbicidal agent. The genetic relationship, in certain inventive embodiments, can be a phylogenetic or other biotype-grouping relationship. In some embodiments, the selected chemical herbicidal agent is the at least one chemical herbicidal agent. The other watermilfoil plants can include Eurasian watermilfoil, North American watermilfoil, and hybrid watermilfoil plants, and/or the confirmed tolerance can be associated with at least some of the hybrid watermilfoil plants.

In another inventive embodiment, provided is a method useful for assisting in the management of a target watermilfoil plant population in a body of water. The method includes obtaining genetic information from one or more samples from the target watermilfoil plant population. The method also includes inferring from the genetic information a genetic relationship of the target watermilfoil plant to other watermilfoil plants, where at least some of the other watermilfoil plants are hybrid watermilfoil plants (typically Northern watermilfoil or Myriophyllum sibiricum×Eurasian watermilfoil or M. spicatum) having confirmed tolerance to at least one chemical herbicidal agent. The genetic relationship, in certain inventive embodiments, can be a phylogenetic or other biotype-grouping relationship. The method can also include generating a visible display of the genetic relationship including indicia representing the target watermilfoil plant and the other watermilfoil plants.

In methods and systems described above and elsewhere herein, the genetic information can, for example, include amplified fragment length polymorphism (AFLP) data and/or simple sequence repeat (SSR) data and/or other molecular marker data. Also, the genetic information can be obtained by receiving a container containing the one or more samples, extracting DNA from the one or more samples, and obtaining the genetic information from the extracted DNA. Any or all of these data can be compared among a target watermilfoil plant and other differing watermilfoil plants. The other differing watermilfoil plants can include, but are not limited to, milfoil species/lineages such as Eurasian watermilfoil, Northern watermilfoil, and hybrid watermilfoils (for instance various Northern×Eurasian lineages or other lineages including crosses with variable watermilfoil (M. heterophyllum) or other watermilfoil species). The chemical herbicide tolerance can be associated with at least some of the hybrid (e.g. Northern×Eurasian) watermilfoil plants. The confirmed chemical herbicidal tolerance can be tolerance to one or more of auxin-mimic herbicides (including but not limited to 2,4-dichlorophenoxyacetic acid (2,4-D), triclopyr), bleaching mode of action herbicides (including but not limited to fluridone and/or another phytoene desaturase inhibitor, or other bleaching herbicide modes of action such as HPPD (hydroxyphenylpyruvate dioxygenase) inhibition), Photosystem I inhibitor herbicides (including but not limited to diquat dibromide), Protox inhibiting herbicides (including but not limited to carfentrezone or flumioxazin), ALS (acetolactate synthase) inhibitor herbicides (including but not limited to penoxsulam, bispyribac, imazamox, or imazapyr), and other potential aquatic herbicide modes of action including non-classified modes of action (e.g., endothall) or future registered aquatic herbicide modes of action. The methods or systems can involve or be useful in the selection of which chemical herbicide to use, and/or a concentration of a chemical herbicide to use, and/or a chemical herbicide contact duration to use, in the control of the target watermilfoil plants. This can for example be determined based on the relationship of the target watermilfoil plants to the known tolerant plants, e.g. whether the target watermilfoil is most closely related to plants known to be tolerant to the one or more chemical herbicides, e.g. in a phylogenetic cluster or other biotype group associated with such tolerance.

Additional embodiments as well as features and advantages of the invention will be apparent from the descriptions herein.

### DESCRIPTION OF THE FIGURES

FIG. 1 depicts a phylogenetic tree including Northern, Eurasian and hybrid (Northern×Eurasian) watermilfoil plants and including a closely related grouping of hybrid (Northern×Eurasian) watermilfoil plants confirmed to exhibit tolerance to multiple chemical herbicides.

FIG. 2 depicts aboveground biomass changes of an auxin herbicide sensitive Eurasian milfoil biotype (A) versus a hybrid milfoil population displaying auxin tolerance (B) at 6 weeks following applications of various rates of 2,4-d and triclopyr. Error bars are 1 SE and letters indicate statistically different treatments per one-way ANOVA and Student Neuman-Keuls method (p=0.05). (reproduced from Glomski and Netherland 2010, Journal of Aquatic Plant Management 48:12-14)

FIG. 3 depicts fluridone herbicide response of a tolerant hybrid milfoil lineage from an Upper Midwest US lake compared to response of a susceptible reference Eurasian milfoil lineage (EWM) in a 14-day laboratory apical tissue assay. Results show less beta-carotene reduction for the tolerant hybrid lineage versus the reference Eurasian lineage at various rates of fluridone, supporting tolerance of the hybrid lineage to the carotenoid synthesis inhibiting herbicide. Error bars are +1 standard deviation (n=4).

FIG. 4 provides a flowchart depicting steps and components in certain embodiments of methods and systems of the invention.

### DETAILED DESCRIPTION

For the purpose of promoting an understanding of the principles of the invention, reference will now be made to certain embodiments thereof and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

As disclosed above, aspects of the present invention relate to methods and systems, and components thereof, to treat bodies of water to control watermilfoil or other similar aquatic weeds growing therein.

In one feature, it has been discovered that watermilfoil populations exhibiting reduced susceptibility (i.e. tolerance) to 2,4-d, fluridone and other chemical herbicides cluster together in phylogenetic trees or other similar comparisons (e.g. biotype groups) and are identified as genetically similar hybrid (Northern×Eurasian) watermilfoil plants. Thus, analysis of a target watermilfoil population in relation to such a phylogenetic tree or another genetic comparison can be used as a predictive tool for herbicide susceptibility of the target watermilfoil population.

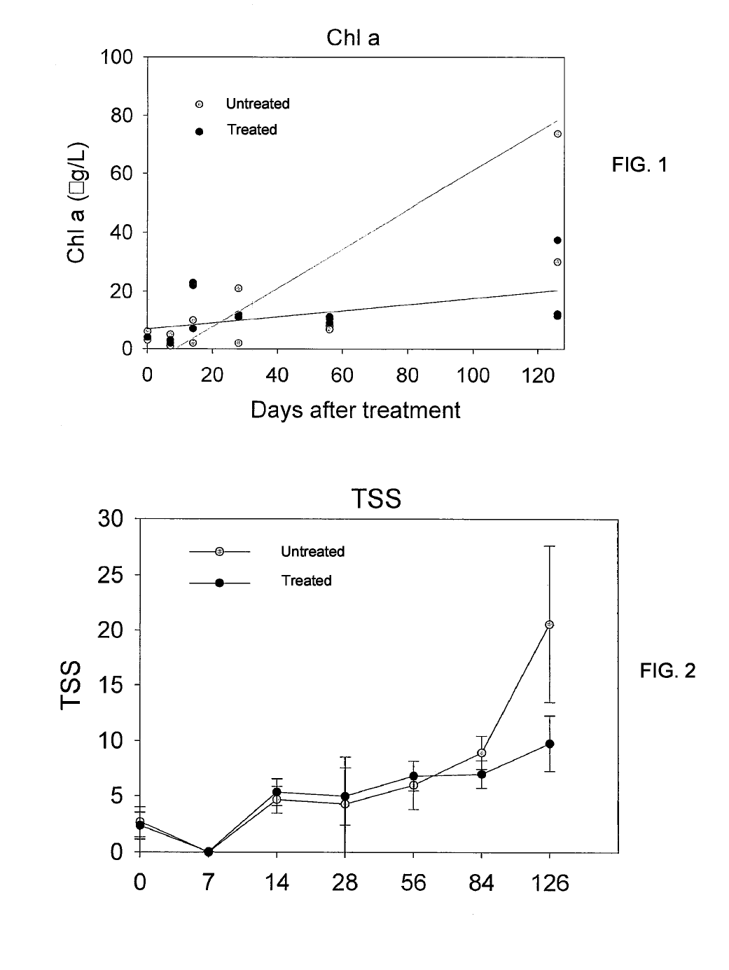

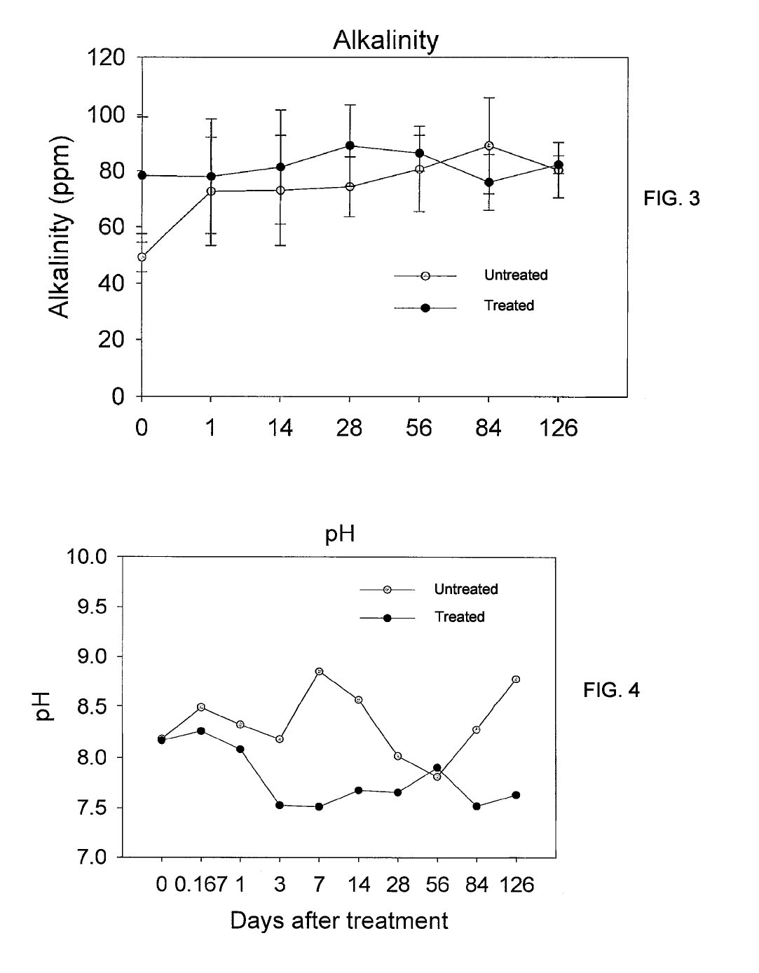

Illustratively, FIG. 1 depicts a computer-generated phylogenetic tree of watermilfoil plants including Northern watermilfoil, Eurasian watermilfoil, and hybrid (Northern×Eurasian) watermilfoil. The tree was generated from AFLP data from samples of watermilfoil populations occurring in the northern region of the United States. Grouped together in the middle of FIG. 1 are several hybrid (Northern×Eurasian) watermilfoil plants (shown with stars) that have been confirmed to exhibit tolerance to either 2,4-d or fluridone aquatic herbicides (see also examples in FIGS. 2 and 3). The phylogenetic relationship of a new target plant to those in the tree of FIG. 1, or to those in another similarly generated phylogenetic comparison, can be used to predict the existence or absence of herbicidal tolerance in the new target plant. Phylogenetic tree-building software suitable for these purposes is commercially available, including for example as PAUP version 4.0 (Sinauer Associates, Inc. Publishers) or TREECON (Yves Van de Peer, Department of Biochemistry University of Antwerp, Belgium), and methods for using such software are likewise known (see e.g. Thum et al., Lake and Reservoir Management 22(1):1-6 (2006)).

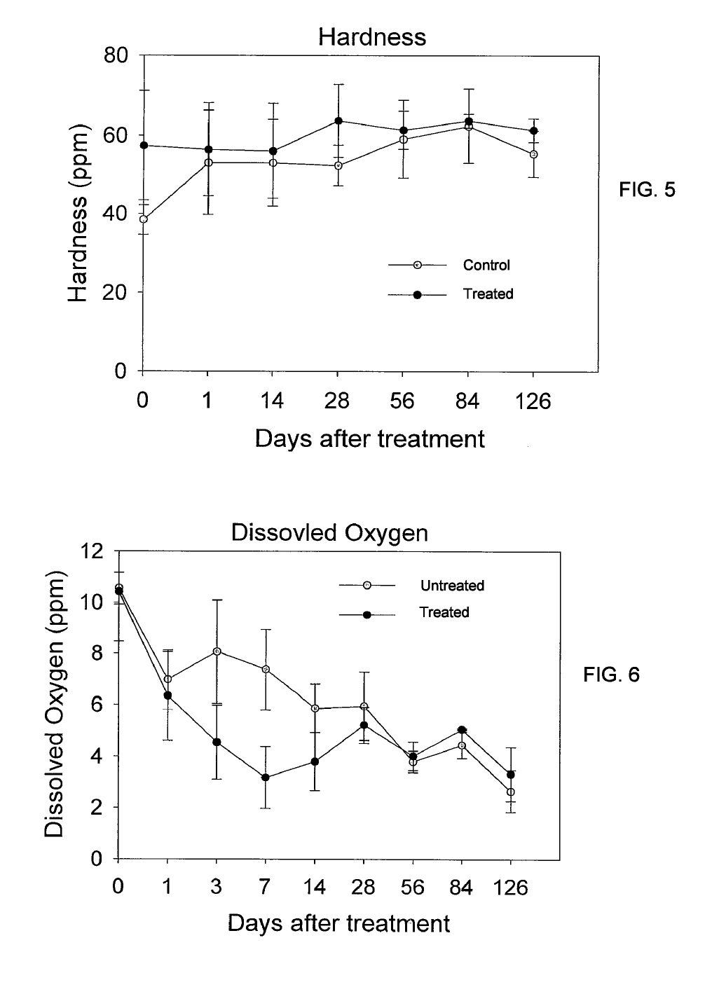

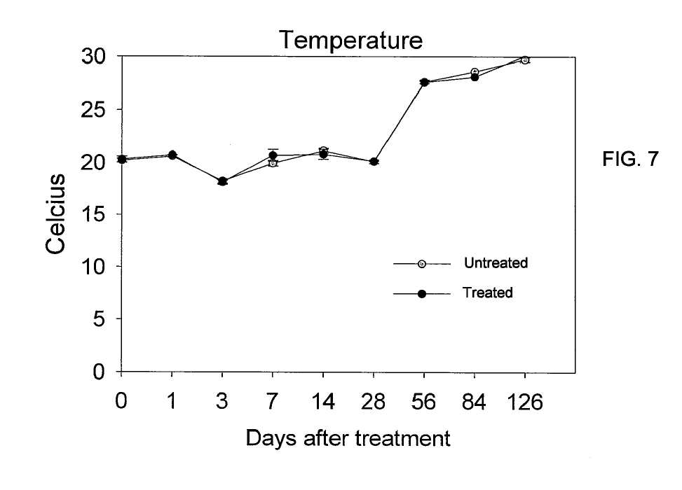

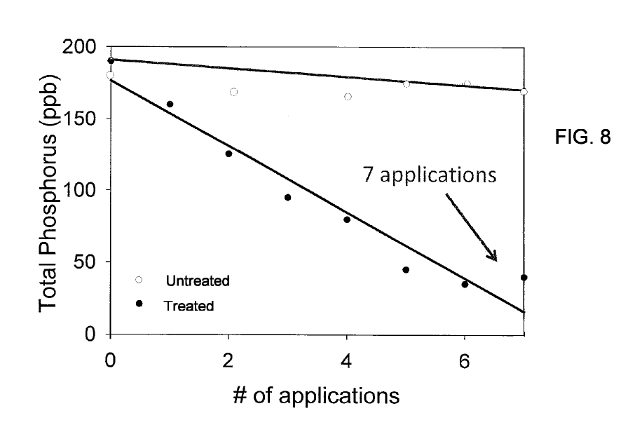

With reference to FIG. 4, shown is a schematic diagram depicting methods and systems for managing watermilfoil plant populations using predictive genetic tools as described herein. In one mode of managing target watermilfoil plants in a lake (10), samples of the plants (11) are obtained and packaged, e.g. in a container, to preserve the samples as appropriate for subsequent testing. The packaged plant samples (11) are shipped to a processing facility (12), such as by overnight courier. The processing facility (12) processes the samples to extract DNA (13), followed by obtaining genetic information from the extracted DNA (14). The thus obtained genetic information is input to a processor (15) along with stored genetic information (16) from previously-characterized watermilfoil plants, at least some of which are identified as herbicide-tolerant hybrid (Northern×Eurasian or other) watermilfoil plants. The computer processor runs a routine to generate a genetic comparison, such as a phylogenetic tree, showing genetic relationships among the target watermilfoil plants and the previously-characterized watermilfoil plants. A visible display (17) is generated, such as a printout or electronic image, depicting the genetic comparison (for example a phylogenetic tree). This display is transmitted to an entity (e.g., a product technical support specialist, a treatment service provider or the lake owner or manager) responsible for developing herbicide treatment recommendations or otherwise involved in the management of the watermilfoil in the lake (10). In one optional embodiment, the processing facility (12) also designs a recommended herbicidal treatment (18) based, at least in part, upon the generated phylogenetic comparison. The recommended herbicidal treatment (18) can be represented in a visible display and transmitted, alone or in addition to the phylogenetic comparison (17), to the lake treating or managing entity.

For the purpose of promoting a further understanding of aspects of the present invention, as well as features and advantages thereof, the following specific Examples are provided. It will be understood that these Examples are illustrative, and not limiting, of embodiments of the invention.

### Example 1

### Watermilfoil Sample Collection and AFLP Analysis

Samples of 28 watermilfoil plant populations were collected from 19 lakes located in Wisconsin and Michigan, USA. All plants were washed thoroughly in distilled water in order to remove/reduce any potential contaminant DNA from symbiotic organisms such as periphyton, insects, snails, etc. Total genomic DNA was extracted from fresh submerged vegetative meristem tissue using DNeasy Plant Mini Kits (Qiagen), similar extraction kits, or a hexadecyltrimethylammonium bromide (CTAB) protocol (Doyle and Doyle 1987. A rapid DNA isolation procedure for small quantities of fresh leaf tissue. Phytochemical Bulletin of the Botanical Society of America 19:11-15). AFLP reactions were prepared as described in Vos et al. (1995), Nucleic Acids Research, Volume 23, Issue 21, pp. 4407-4414, with some modifications.

For the restriction digestion, ˜100 ng of total genomic DNA was digested with EcoRI and MseI restriction enzymes using the following: 5 units EcoRI enzyme, 1 unit MseI enzyme, 4 μL 10× T4 DNA ligase buffer with ATP (New England Biolabs), 4 μL NaCl (0.5M), 2 μL BSA (1 mg/mL), and water to a final volume of 40 uL. Reactions were incubated at 37° C. for one hour.

For Adapter ligation, the EcoRI and MseI adaptors were ligated by adding the following to the digested genomic DNA: 1 unit T4 DNA ligase, 1 μL 10× T4 DNA ligase buffer with ATP, 1 μL NaCl (0.5M), 0.54 BSA (1 mg/mL), 1 μL annealed EcoRI adaptors (Applied Biosystems), 1 μL annealed MseI adaptors (Applied Biosystems), and water to a final volume of 50 μL. Reactions were incubated at 37° C. for three hours.

Preselective PCR amplification employed primers EcoRI-A and MseI-C and therefore amplified only those digested segments that contained a 3′ A or C on the EcoRI and MseI ends of the restriction-ligation fragments, respectively. The preselective amplification reactions consisted of: 1 μL preselective primers (Applied Biosystems), 15 μL AFLP Core Mix (Applied Biosystems), and 4 μL restriction-ligation product (after diluting five-fold in water). Preselective thermal cycling conditions consisted of one cycle at 72° C. for 2 minutes, followed by 20 cycles of: 94° C. for 2 min; 56° C. for 30 s, and 72° C. for 2 min; with a final extension at 60° C. for 30 minutes. Preselective products were run out on an agarose gel (˜1%) to ensure that amplification had occurred.

Selective PCR amplification was employed to add a fluorescent label to the EcoRI fragments and further limit the number of digested products amplified by employing primers that added an additional two nucleotides to the 3′ end of the preselective primers.

The current work used only one pair of primers (EcoRI-ACA and MseI-CAT), but additional primers could also be used. Selective amplifications consisted of: 1 μL of each selective primer, 15 μL AFLP Core Mix, and 3 μL preselective product (after 20-fold dilution in water). Reaction conditions for the selective amplification consisted of one cycle at 94° C. for 20 seconds, 66° C. for 30 seconds, and 72° C. for 2 minutes; the annealing temperature was then lowered 1° C. each cycle during the next 10 cycles (i.e., 56° C. in the tenth cycle). Twenty additional cycles were performed using an annealing temperature of 56° C., followed by a final extension at 60° C. for 30 minutes. Selective amplification products were run on an ABI 3130xl automated DNA sequencer at AWRI using the internal size standard MapMarker1000 ROX (BioVentures, Inc.).

### Example 2

### Analysis of AFLP Data

### A. GeneMapper

AFLP genotype data were scored with GeneMapper v4.0 (Applied Biosystems). The analysis was limited to fragments between 80 and 500 bp in length. The binset was constructed using a peak height threshold (PHT) of 200 relative fluorescence units (rfu) and a bin width of 0.75 bp. Each sample was then automatically scored for this binset using a PHT of 30 rfu. All allele calls were also visually checked and edited.

### B. Structure

Structure v2.3.2 (Pritchard et al., Inference of population structure using multilocus genotype data, Genetics, 155:945-959 (2000); Falush et al., Inference of population structure using multilocus genotype data: dominant markers and null alleles, Molecular Ecology Notes (2007)) was used to identify genetically distinct groups (biotypes) and individual membership to these groups. Briefly, Structure uses an iterative Bayesian Markov Chain Monte Carlo (MCMC) method to simultaneously evaluate the number of distinct genetic groups within a dataset (K) and assign a proportion of each individual's genome to each of the K distinct groups. The number of distinct genetic groups in a dataset is evaluated by comparing likelihood scores for runs at different values of K whereas the proportion of an individual's genome attributed to each value of K is determined by the posterior probability of membership to each group.

Structure can employ a variety of different models for population structure, including allowing for admixture (hybridization) among different groups, correlated allele frequencies among groups, and estimating the admixture proportion (a) separately for each group. Models employing all possible combinations of the above parameters were evaluated. A typical run of the MCMC was >75,000 generations, preceded by a burn-in period of 25,000 generations. Likely values of K for the dataset were identified by (i) graphing −ln likelihood of data vs. K, (ii) determining which values of K consistently contained individuals with high assignment probabilities, and (iii) evaluating clusters identified with AFLP in the context of patterns identified with inter- and intra-specific genetic variation in ITS.

### C. Discussion: Results of Genetic Analyses

The results of comparative genetic analyses, which can be generated with the assistance of well known commercial software (e.g. for generating phylogenetic tree-based outputs), are displayed in FIGS. 1, 2, and 3. In FIG. 1, lakes with stars beside them have either compelling anecdotal evidence or confirmed tolerance per quantitative testing (see FIGS. 2 and 3) for reduced susceptibility to the herbicides 2,4-d and fluridone. The shaded dots correspond to Structure, K=6, shown to the right. A 10% distance corresponds to 9.7 AFLP bands.

As can be seen, it has been discovered that confirmed or highly suspect herbicide tolerant hybrid (Northern×Eurasian) watermilfoil plants occur in a highly phylogenetically related cluster. Correlation of herbicide tolerance to relative position on the phylogenetic tree enables the use of genetic molecular analyses of target watermilfoil populations to predict susceptibility to a given herbicide(s), and thus the design of a treatment regimen taking into account potential tolerances in the target watermilfoil populations. A new target watermilfoil population can be sampled, and genetic data such as those described above can be obtained and used to infer a position of the new target population on the phylogenetic tree of FIG. 1 or a derivative thereof, or another generated phylogenetic tree with correlated tolerance information. A treatment regimen using a chemical herbicide can then be designed based at least in part on this analysis, and carried out to control the watermilfoil population. In an additional model for assessment and treatment planning, placement of a new target watermilfoil population in a phylogenetic group with documented risk for tolerance to one or more herbicides would form the basis for implementing focused, laboratory-scale herbicide susceptibility assays of milfoil plants to confirm anticipated plant response to treatment.

All publications cited herein are hereby incorporated by reference in their entirety as if each had been individually incorporated by reference and fully set forth.

The use of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

1. A method for controlling watermilfoil in a lake, comprising:
obtaining samples of target watermilfoil plants living in a lake;
obtaining genetic information from the samples;
inferring from said genetic information a genetic relationship of the target watermilfoil plants to other watermilfoil plants, wherein at least some of the other watermilfoil plants have confirmed tolerance to at least one chemical herbicidal agent; and
treating the body of water with a selected chemical herbicidal agent in accordance with a treatment regimen based at least in part upon said inferring step.

2. The method of claim 1, wherein said genetic information comprises comparing molecular marker information.

3. The method of claim 1, wherein said obtaining genetic information includes obtaining amplified fragment length polymorphism (AFLP) data from the samples, and said inferring comprises comparing said AFLP data to AFLP data from the other watermilfoil plants.

4. The method of claim 1 wherein the selected chemical herbicidal agent is auxin-mimic herbicide.

5. The method of claim 1, wherein said at least one chemical herbicidal agent is 2,4-D and/or triclopyr.

6. The method of claim 1 wherein the selected chemical herbicidal agent is a phytoene desaturase inhibitor.

7. The method of claim 1 wherein said selected chemical herbicidal agent is fluridone.

8. The method of claim 7, wherein said at least one chemical herbicidal agent is fluridone, and wherein said treatment regimen includes a target fluridone concentration in the lake based at least in part upon said inferring step.

9. The method of claim 1, wherein the other watermilfoil plants in the phylogenetic tree include Eurasian watermilfoil, Northern watermilfoil, and/or hybrid watermilfoils.

10. The method of claim 9, wherein the hybrid watermilfoil plants include at least some of said watermilfoil plants having confirmed tolerance to at least one chemical herbicidal agent.

11. A system useful for inferring a phylogenetic relationship of a target watermilfoil plant to other watermilfoil plants, the system comprising:
a processing unit;
one or more memory storage units coupled to said processing unit, the one or more memory storage units storing (i) a routine for estimating a genetic relationship, (ii) first data representing genetic information from the target watermilfoil plant, and (iii) second data representing genetic information from differing watermilfoil plants and associating confirmed chemical herbicidal resistance with at least some of said multiple differing watermilfoil plants; and
wherein the routine, first data and second data are processable by the processing unit to estimate a genetic relationship between the target watermifoil plant and the genetically differing watermilfoil plants.

12. The system of claim 11, wherein said first and second data comprise amplified fragment length polymorphism (AFLP) data.

13. The system of claim 11, wherein first and second data comprise molecular marker data.

14. The system of claim 11, wherein the chemical herbicidal resistance tolerance to a phytoene desaturase inhibitor.

15. The system of claim 14, wherein the chemical herbicidal tolerance is tolerance to fluridone.

16. The system of any of claim 11, wherein the chemical herbicidal tolerance is tolerance to 2,4-D.

17. The system of claim 11, wherein the chemical herbicidal tolerance is tolerance to triclopyr.

18. The system of claim 11, wherein the genetically differing watermilfoil plants include Eurasian watermilfoil, North American watermilfoil, and hybrid watermilfoil plants.

19. The system of claim 18, wherein the chemical herbicidal tolerance is associated with at least some of the hybrid watermilfoil plants.

20. A method for controlling target watermilfoil plants in a body of water, comprising:
treating the body of water with a selected chemical herbicidal agent in accordance with a treatment regimen, wherein said treatment regimen has been based at least in part upon inferring from genetic information from said target watermilfoil plant a genetic relationship of the target watermilfoil plants to other watermilfoil plants, wherein at least some of the other watermilfoil plants have confirmed tolerance to at least one chemical herbicidal agent.

21. The method of claim 20, wherein said selected chemical herbicidal agent is said at least one chemical herbicidal agent.

22. The method of claim 20, wherein the other watermilfoil plants include Eurasian watermilfoil, North American watermilfoil, and hybrid watermilfoil plants.

23. The method of claim 22, wherein the confirmed tolerance is associated with at least some of the hybrid watermilfoil plants.

24. The method of claim 20, wherein said genetic information comprises amplified fragment length polymorphism (AFLP) data.

25. The method of claim 20, wherein said genetic information comprises molecular marker data.

26. A method useful for assisting in the management of a target watermilfoil plant population in a body of water, comprising:
obtaining genetic information from one or more samples from the target watermilfoil plant population;
inferring from said genetic information a genetic relationship of the target watermilfoil plants to other watermilfoil plants, wherein at least some of the other watermilfoil plants are hybrid watermilfoil plants having confirmed tolerance to at least one chemical herbicidal agent.

27. The method of claim 26, wherein said genetic information comprises molecular marker data.

28. The method of claim 26, wherein said obtaining genetic information comprises receiving a container containing the one or more samples, extracting DNA from the one or more samples, and obtaining the genetic information from the extracted DNA.

29. The method of claim 26, also comprising generating a visible display of the genetic relationship including indicia representing the target watermilfoil plant and the other watermilfoil plants.

30. A method according to claim 1, wherein the genetic relationship is an association of the target plant with a biotype group of the other watermilfoil plants
